(19) United States
(12) Patent Application Publication
(10) Pub. No.: US 2018/0000500 A1
(43) Pub. Date: Jan. 4, 2018

(54) FLEXIBLE-MANIPULATOR CONTROL DEVICE AND MEDICAL MANIPULATOR SYSTEM

(71) Applicant: OLYMPUS CORPORATION, Tokyo (JP)

(72) Inventor: Naoya HATAKEYAMA, Tokyo (JP)

(73) Assignee: OLYMPUS CORPORATION, Tokyo (JP)

(21) Appl. No.: 15/706,978

(22) Filed: Sep. 18, 2017

Related U.S. Application Data
(63) Continuation of application No. PCT/JP2015/058955, filed on Mar. 24, 2015.

(51) Int. Cl. A61B 17/28; B25J 9/10; B25J 9/16; B25J 18/06

(52) U.S. Cl. CPC A61B 17/28; B25J 18/06; B25J 9/10; B25J 9/1602

(57) ABSTRACT

A flexible-manipulator control device that controls, according to a control parameter, a drive of a flexible manipulator having a movable part at a distal end of a flexible insertion portion to be inserted into the body and having, at a proximal end thereof, the drive for driving the movable part, includes: a physical-information storage unit that stores physical information of a patient into which the insertion portion is inserted; a position-information input unit to which is input position information of the movable part in a state in which it is inserted into the patient; and a parameter adjustment unit that adjusts the control parameter on the basis of the physical information stored in the physical-information storage unit and the position information input to the position-information input unit.

### CROSS-REFERENCE TO RELATED APPLICATIONS

This is a continuation of International Application PCT/JP2015/058955 which is hereby incorporated by reference herein in its entirety.

### TECHNICAL FIELD

The present invention relates to a flexible-manipulator control device and a medical manipulator system.

### BACKGROUND ART

There is a known technique for changing, in a flexible endoscope, control parameters for a treatment tool according to the curved state of an insertion portion (for example, see PTL 1). In PTL 1, the tensile force of a wire for driving bending of a curved portion at the distal end of an insertion portion is detected, thereby detecting the curved state of the insertion portion.

Furthermore, there is also a known technique for using a UPD device in order to detect the curved state of the insertion portion (for example, see PTL 2).

### CITATION LIST

### Patent Literature

{PTL 1} Publication of Japanese Patent No. 4580973
{PTL 2} Japanese Unexamined Patent Application, Publication No. 2009-131406

### SUMMARY OF INVENTION

According to one aspect, the present invention provides a flexible-manipulator control device that controls, according to a control parameter, a drive means of a flexible manipulator having a movable part at a distal end of a flexible insertion portion to be inserted into the body and having, at a proximal end thereof, the drive means for driving the movable part, the flexible-manipulator control device including: a physical-information storage unit that stores physical information of a patient into which the insertion portion is inserted; a position-information input unit to which is input position information of the movable part in a state in which it is inserted into the patient; and a parameter adjustment unit that adjusts the control parameter on the basis of the physical information stored in the physical-information storage unit and the position information input to the position-information input unit.

### BRIEF DESCRIPTION OF DRAWINGS

FIG. 1 is a view showing the overall configuration of a medical manipulator system according to one embodiment of the present invention.

FIG. 2 is a block diagram showing a flexible-manipulator control device according to the one embodiment of the present invention, provided in the medical manipulator system shown in FIG. 1.

FIG. 3 is a view showing an example shape of a flexible manipulator when inserted into the large intestine.

FIG. 4 is a view showing a state in which the shape of the flexible manipulator shown in FIG. 3 is modeled with straight lines and circular arcs, and a plurality of sections including curved portions are set.

FIG. 5 is a view showing a modification of the medical manipulator control device shown in FIG. 2.

FIG. 6 is a view showing another modification of the medical manipulator control device shown in FIG. 2.

### DESCRIPTION OF EMBODIMENT

A medical manipulator system 1 and a flexible-manipulator control device 2 according to one embodiment of the present invention will be described below with reference to the drawings.

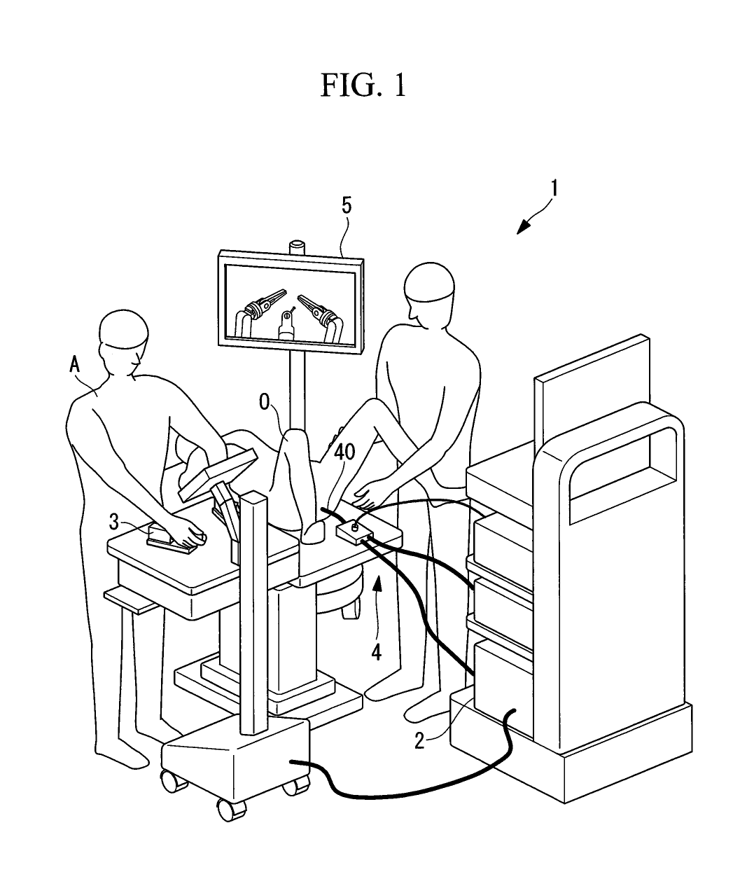

As shown in FIG. 1, for example, the medical manipulator system 1 of this embodiment is provided with: a master device (operation unit) 3 that is operated by an operator A; a flexible manipulator 4 that is to be inserted into a body cavity of a patient O; a flexible-manipulator control device 2 that controls the flexible manipulator 4 on the basis of an operation input that is input to the master device 3; and a monitor 5.

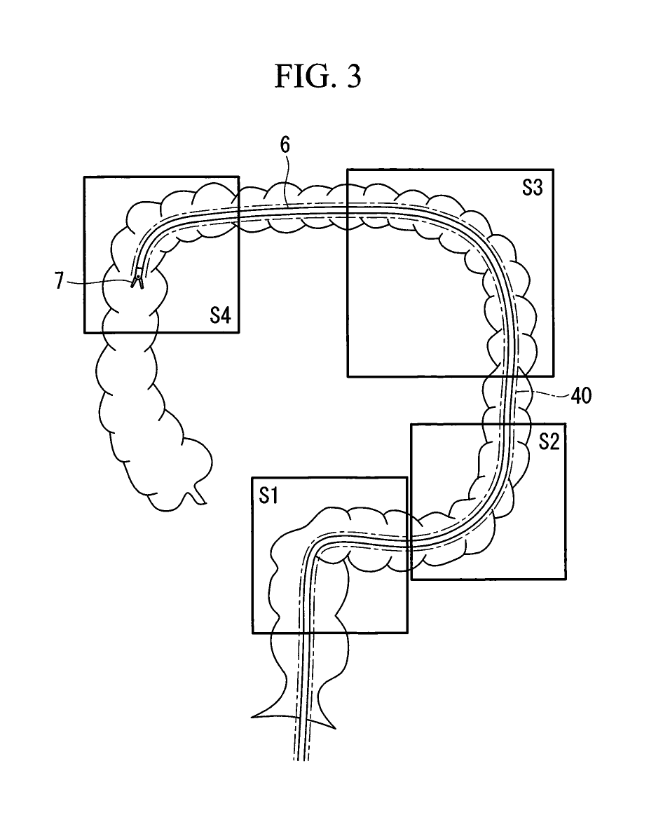

The flexible manipulator 4 is provided with: an insertion portion 6 that is to be inserted into the body cavity of the patient O via a forceps channel of a flexible endoscope, an overtube 40 (see FIG. 3), or the like that is to be inserted into the body cavity of the patient O; a gripping portion (movable part) 7 that is disposed at a distal end of the insertion portion 6; a drive portion (drive means) 8 that is disposed at a proximal end of the insertion portion 6; and a wire 20 that connects the drive portion 8 and the gripping portion 7 and that causes the gripping portion 7 to move due to the tensile force thereof.

The gripping portion 7 is gripping forceps, for example, and is made to perform opening and closing movement due to the tensile force of the wire 20.

The drive portion 8 is provided with a motor 9 and a conversion mechanism 10, such as a pulley, that converts the driving force of the motor 9 into the tensile force of the wire 20.

The wire 20 connects the gripping portion 7 and the drive portion 8 along a path formed in the insertion portion 6.

The flexible-manipulator control device 2 of this embodiment is provided with: an identification-information input unit 11 to which identification information of the patient O is input; a storage unit (physical-information storage unit) 12 that stores the identification information of the patient O and physical information thereof in association with each other; a selection unit (physical-information selection unit) 13 that selects physical information of the patient O related to identification information input to the identification-information input unit 11; a position-information input unit 14 to which is input position information of the gripping portion 7 in a state in which it is inserted into the body cavity of the patient O; a drive control unit 15 that controls the drive portion 8 in response to an operation input that is input to the master device 3; and a parameter adjustment unit 16 that adjusts a control parameter in the drive control unit 15 on the basis of the physical information selected by the selection unit 13 and the position information input to the position-information input unit 14.

Physical information of the patient O can include information indicating an organ shape of the patient O, in particular, the shape of an organ having a body cavity, which serves as an insertion path into which the insertion portion 6 is inserted. For example, the physical information includes BMI, height, weight, nationality, sex, age, body type, surgery history, etc. of the patient O. These items of physical information indirectly indicate the state of the body cavity, which serves as an insertion path into which the insertion portion 6 is inserted, for example, the diameter, the length, the curvature, etc. of the body cavity.

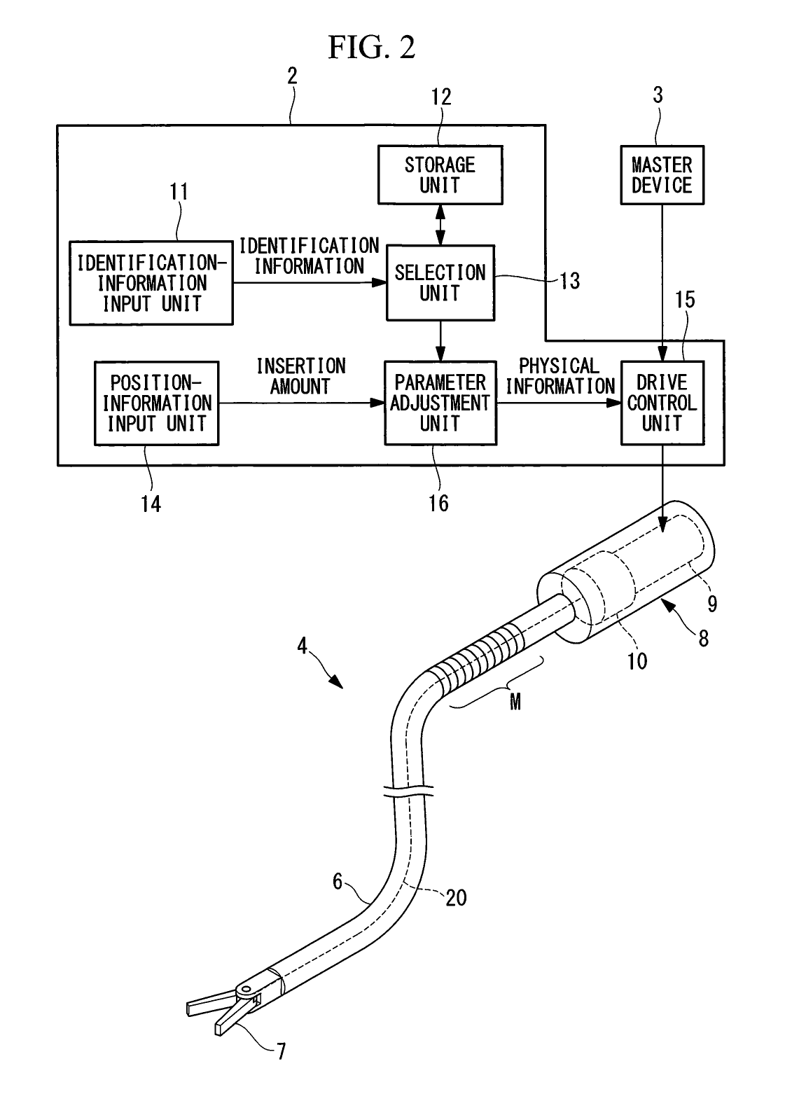

The position-information input unit 14 is an input device to which the operator A inputs an insertion amount of the insertion portion 6 into the body cavity. The insertion amount of the insertion portion 6 into the body cavity can be easily confirmed outside the body of the patient O, by providing in advance a scale M on the outer surface of the insertion portion of the endoscope or the overtube 40, into which the insertion portion 6 is inserted. If the flexible manipulator 4 is directly inserted into the body cavity, the scale M may be provided in advance on the insertion portion 6 of the flexible manipulator 4, to confirm the insertion amount with the scale M, as shown in FIG. 2.

The drive control unit 15 holds, in the initial state, a control parameter that causes the motor 9 of the drive portion 8 to generate a necessary and sufficient driving force for opening and closing the gripping portion 7 in response to an operation input that is input to the master device 3, in a state in which the insertion portion 6 extends straight.

The parameter adjustment unit 16 adjusts the control parameter in the drive control unit 15 in the following procedure.

Specifically, the shape of the organ having the body cavity, which serves as an insertion path into which the insertion portion 6 is inserted, can be identified by the physical information of the patient O selected by the physical-information selection unit 13, and, when an insertion amount is input, the position in the organ that the gripping portion 7, which is disposed at the distal end of the insertion portion 6, reaches can be estimated.

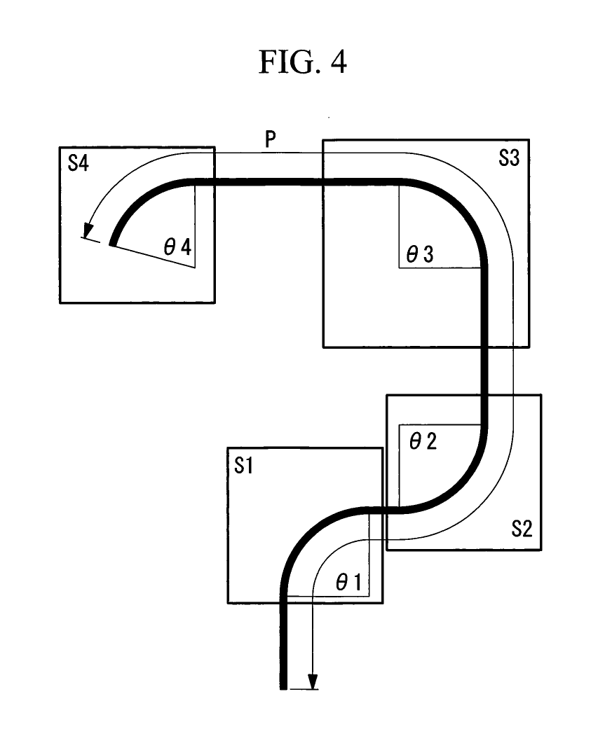

It is assumed that the actual shape of the insertion portion 6 inserted into the organ, as shown in FIG. 3, is the shape expressed by a combination of straight lines and circular arcs having certain curvatures, as shown in FIG. 4. In the example shown in FIG. 3, the large intestine is shown as an organ into which the flexible manipulator 4 is inserted. The position of the gripping portion 7, which is disposed at the distal end of the insertion portion 6, is set using an insertion amount P of the insertion portion 6.

Then, as shown in FIG. 3, in the set shape of the insertion portion 6, sections S1 to S4 are set for respective curved portions each of which can be approximated by a single circular arc. Thereafter, bend angles θ1 to θ4 of the curved portions of the insertion portion 6 included in the respective sections S1 to S4 are calculated. In this example, because the gripping portion 7, which is disposed at the distal end of the insertion portion 6, reaches up to the section S4 on the basis of the insertion amount P, the bend angles θ1 to θ3 in the sections S1 to S3 are 90 degrees, and an angle smaller than 90 degrees is calculated from the insertion amount P for the bend angle θ4 in the section S4. Then, the total bend angle θ(=θ1+θ2+θ3+θ4) throughout the length of the insertion portion 6 is calculated.

When the total bend angle θ is calculated in this way, the parameter adjustment unit 16 calculates the control parameter by using the calculated total bend angle θ.

An example control parameter can be the apparent rigidity k_{d} of the wire 20.

The apparent rigidity k_{d} of the wire 20 is expressed by the following expression (1) by using the total bend angle θ.

k_{d}=EAμθ/(L(e^{μθ}−1)) (1)

where,
E is the Young's modulus of the wire 20,
A is the cross-sectional area of the wire 20,
μ is the coefficient of friction between the wire 20 and the surroundings thereof, and
L is the length of the wire 20.

Then, the parameter adjustment unit 16 overwrites the rigidity of the wire 20, which is the control parameter stored in the drive control unit 15, with the apparent rigidity k_{d} calculated by expression (1), so as to output a command signal for driving the gripping portion 7 by using the wire 20 having the apparent rigidity k_{d}.

The operation of the thus-configured flexible-manipulator control device 2 and medical manipulator system 1 of this embodiment will be described below.

To treat an affected area located in the body cavity of the patient O by using the medical manipulator of this embodiment, the operator A first inputs the identification information of the patient O via the identification-information input unit 11.

When the identification information of the patient O is input, the selection unit 13 searches for the physical information of the patient O stored in the storage unit 12 on the basis of the identification information and selects the physical information associated with this identification information. The selected physical information is sent to the parameter adjustment unit 16.

Next, the operator A inserts the endoscope or the overtube 40 into the body cavity of the patient O and makes it move up to a position where the distal end of the endoscope or the overtube 40 faces the affected area.

Since the endoscope or the overtube 40 has a relatively large rigidity, it is curved while following the shape of the organ, whereas it is inserted while deforming the organ at a section of the organ where the rigidity is weak.

Then, the operator A confirms the insertion amount of the insertion portion of the endoscope or the overtube 40 by means of the scale M, which is provided on the outer surface of the endoscope or the overtube 40, and inputs the insertion amount via the position-information input unit 14. The insertion amount input via the position-information input unit 14 is also sent to the parameter adjustment unit 16.

In the parameter adjustment unit 16, the shape of the organ having the body cavity, which serves as an insertion path into which the insertion portion 6 is inserted, is identified on the basis of the physical information selected by the selection unit 13. Then, the position in the shape of the organ up to which the insertion portion 6 has been inserted can be identified on the basis of the insertion amount input via the position-information input unit 14.

Accordingly, in the parameter adjustment unit 16, the shape of the insertion portion 6 being inserted into the body cavity is estimated.

Then, in the parameter adjustment unit 16, the estimated shape of the insertion portion 6 is divided into the plurality of sections S1 to S4 for the respective curved portions each of which can be approximated by a single circular arc, and the bend angles of the curved portions included in the sections S1 to S4 are added up, thus calculating the total bend angle θ throughout the length of the insertion portion 6.

Then, in the parameter adjustment unit 16, the apparent rigidity k_{d} of the wire 20 is calculated with expression (1) by using the total bend angle θ, the calculated rigidity k_{d} is sent to the drive control unit 15, and the stored rigidity is replaced with the calculated rigidity k_{d}. Accordingly, the control parameter for the drive portion 8 for driving the gripping portion 7 via the wire 20 is replaced with a realistic value.

Specifically, as the curved portion of the insertion portion 6 caused by inserting the insertion portion 6 into the body cavity becomes longer, the wire 20 is influenced by friction, thus making it difficult to transfer a tensile force given by the drive portion 8; however, according to the flexible-manipulator control device 2 and the medical manipulator system 1 of this embodiment, the length of the curved portion is estimated, and the control parameter is overwritten, thereby making it possible to generate a larger driving force to generate a necessary and sufficient tensile force for opening and closing the gripping portion 7. Accordingly, there is an advantage that drive control for the gripping portion 7 performed by the drive portion 8 can be accurately performed.

Furthermore, according to the flexible-manipulator control device 2 and the medical manipulator system 1 of this embodiment, because the total bend angle of the insertion portion 6 is calculated on the basis of the physical information of the patient O and the position information of the gripping portion 7 in the body cavity, the control accuracy for the gripping portion 7 can be easily improved.

In particular, because it is only necessary to manually input the insertion amount of the insertion portion 6 as the position information of the gripping portion 7, there is an advantage that the control accuracy can be improved at low cost, without using an expensive device, such as a UPD device.

Furthermore, in this embodiment, because the identification information of the patient O is input, and the physical information of the patient O is read, the optimal drive control can be performed for each patient O.

Note that, in this embodiment, although, in order to estimate the curved state of the insertion portion 6 of the flexible manipulator 4, the insertion portion 6 is divided into a plurality of sections S1 to S4, and the bend angles θ1 to θ4 calculated for the respective sections S1 to S4 are added up, thus calculating the total bend angle θ, instead of this, it is also possible to adopt other methods, to be described below.

In the first, it is also possible to store a look-up table in which a combination of the physical information and the insertion amount is associated in advance with the control parameter and to read out the control parameter from the look-up table on the basis of input physical information and insertion amount P.

Accordingly, there is an advantage that the control parameter can be rapidly adjusted.

In the second, it is also possible to prepare the control parameter in the form of a function in which the physical information and the insertion amount P are used as arguments and to substitute input physical information and insertion amount P into the function, thus calculating the control parameter.

With this method, there is also an advantage that the control parameter can be rapidly adjusted.

As the function, it is also possible to adopt a function in which, as the physical information, the organ shape is assumed to be “circular arcs” having different radii according to the patient O, and the bend angle is output by using the insertion amount as the length of the circular arc. According to this, the control parameter can be rapidly calculated with a simple function.

Furthermore, as the function, it is also possible to adopt a free function that expresses a line segment having a shape closer to an actual organ shape. According to this, the curved state of the insertion portion 6 can be more accurately expressed.

Furthermore, although the total bend angle θ is calculated on the assumption that the all curved portions have a certain curvature, instead of this, it is also possible to set different curvatures in the respective curved portions so as to more faithfully express the shape of the organ and to calculate the circular arc length of the curved portions from the curvatures and the bend angles.

Furthermore, although the insertion amount is input as the position information, for example, in a case in which the position of the gripping portion 7 can be confirmed by the endoscope, the position thereof may be directly identified.

Furthermore, although the insertion amount is read by means of the scale M, instead of this, it is also possible to dispose a sensor for detecting the movement of the insertion portion 6 in the longitudinal direction and to detect, with the sensor, the amount of movement since insertion of the insertion portion 6 into the body cavity was started.

As the sensor, it is possible to adopt a rotation sensor that detects the rotation angle of a roller that is made to roll on the outer surface of the insertion portion 6, a Hall element that detects magnetism of magnets fixed to the insertion portion 6 at intervals in the longitudinal direction, or a capacitance sensor.

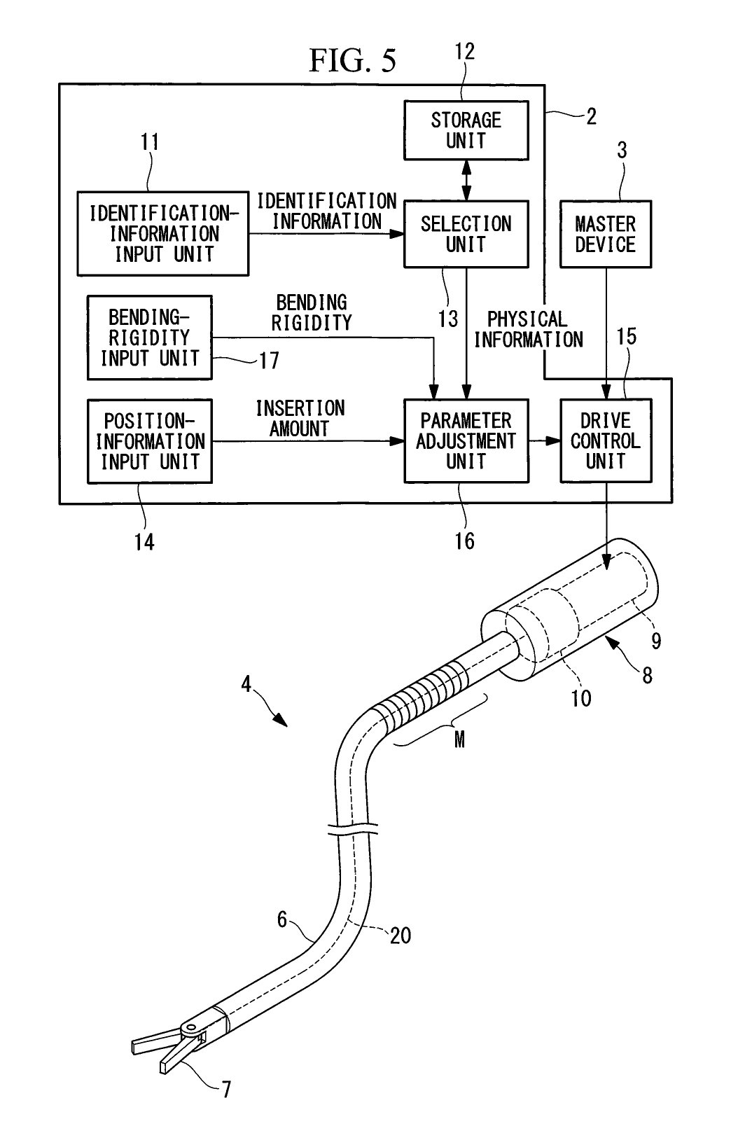

Furthermore, although the control parameter is calculated from the physical information and the insertion amount P in the parameter adjustment unit 16, in addition to this, as shown in FIG. 5, a bending rigidity of the insertion portion 6 may be input via a bending-rigidity input unit 17 and may be used to calculate the control parameter. Because the flexible manipulator 4 is influenced by the bending rigidity when it is curved, the state of the flexible manipulator 4 can be estimated with higher accuracy.

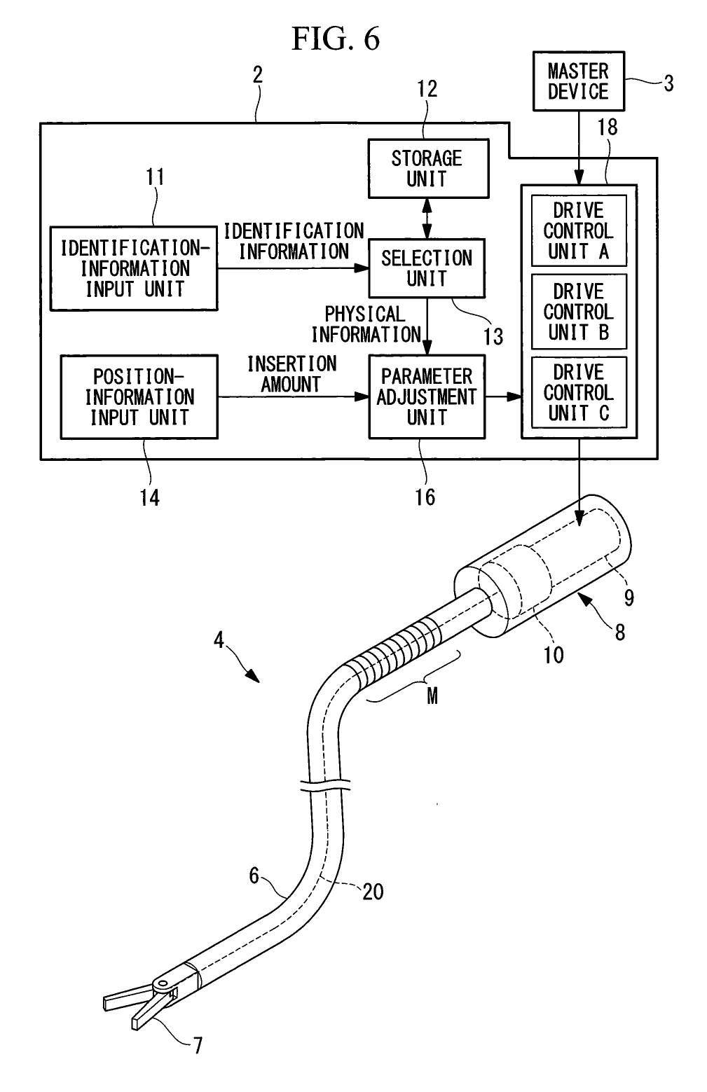

Furthermore, instead of replacing the control parameter, as shown in FIG. 6, it is also possible to provide a plurality of drive control units (drive control units A, B, and C, control units) 18 that hold different control parameters, in a switchable manner, and to selectively connect one of the drive control units A, B, and C according to a combination of the physical information and the insertion amount. In addition to a case in which the control parameter is switched in the same control model, it is also possible to adopt a different control model, thus making it possible to control the gripping portion 7 with higher accuracy.

Furthermore, adjustment of the control parameter may be performed every time the operator A instructs calibration or may be performed successively according to the insertion amount of the insertion portion 6. Through the adjustment in a short cycle, control can be performed with higher accuracy.

The above-described embodiment leads to the following invention.

According to one aspect, the present invention provides a flexible-manipulator control device that controls, according to a control parameter, a drive means of a flexible manipulator having a movable part at a distal end of a flexible insertion portion to be inserted into the body and having, at a proximal end thereof, the drive means for driving the movable part, the flexible-manipulator control device including: a physical-information storage unit that stores physical information of a patient into which the insertion portion is inserted; a position-information input unit to which is input position information of the movable part in a state in which it is inserted into the patient; and a parameter adjustment unit that adjusts the control parameter on the basis of the physical information stored in the physical-information storage unit and the position information input to the position-information input unit.

According to this aspect, the control parameter for controlling the drive means, which is disposed at the proximal end of the flexible manipulator, in order to drive the movable part, which is located at the distal end of the flexible manipulator, is adjusted on the basis of the physical information of the patient stored in the physical-information storage unit and the position information of the movable part input to the position-information input unit. Because the physical information shows the shape of the insertion path in the body into which the insertion portion is inserted, and the position information of the movable part shows the insertion length from an insertion hole, the curved degree of the insertion portion can be estimated on the basis of these two pieces of information. As a result, the movable part, which is disposed at the distal end of the curved insertion portion, can be accurately controlled by the drive means, which is disposed at the proximal end of the insertion portion.

In the above-described aspect, the physical-information storage unit may store physical information of a plurality of patients in association with identification information of the patients; the flexible-manipulator control device may further include: an identification-information input unit to which identification information of a patient is input; and a physical-information selection unit that selects the physical information of the corresponding patient stored in the physical-information storage unit, on the basis of the identification information of the patient input to the identification-information input unit; and the parameter adjustment unit may adjust the control parameter on the basis of the physical information selected by the physical-information selection unit and the position information input to the position-information input unit.

By doing so, because the identification information of the patient is input, and the physical information suitable for the patient who undergoes surgery is selected from the plurality of pieces of physical information stored in the physical-information storage unit, it is possible to accurately control the flexible manipulator by adjusting the control parameter appropriately.

Furthermore, in the above-described aspect, the physical information may be information about the shape of an insertion path in which the insertion portion is inserted into the body.

By doing so, on the basis of the information about the shape of the insertion path in which the insertion portion is inserted into the body and the position information of the inserted movable part, it is possible to more accurately estimate the curved state of the insertion portion and to accurately control the movable part.

Furthermore, in the above-described aspect, the position information of the movable part may be an insertion amount of the insertion portion inserted into the body.

By doing so, on the basis of the insertion amount and the physical information, it is possible to more accurately estimate the curved state of the insertion portion and to accurately control the movable part.

Furthermore, in the above-described aspect, the position-information input unit may be provided with a sensor for detecting the insertion amount of the insertion portion.

By doing so, because the insertion amount of the insertion portion is detected by the sensor, and the detected insertion amount is input, it is possible to easily and accurately estimate the curved state of the insertion portion, without performing manual input.

Furthermore, the above-described aspect may further include a plurality of control units in which different control parameters for controlling the drive means are set and that can be alternatively selected, wherein the parameter adjustment unit may switch between the control units on the basis of the physical information and the position information.

By doing so, the control unit in which the optimum control parameter is set is selected on the basis of the physical information of the patient and the position information, thus making it possible to easily and accurately control the movable part.

Furthermore, in the above-described aspect, the parameter adjustment unit may estimate the shape of the insertion portion on the basis of the position information input to the position-information input unit and may adjust the control parameter on the basis of the estimated shape of the insertion portion.

By doing so, because the shape of the insertion portion is estimated on the basis of the physical information of the patient and the position information, it is possible to grasp the curved state of the insertion portion in more detail, thus making it possible to more accurately control the movable part.

Furthermore, according to another aspect, the present invention provides a medical manipulator system including: the flexible manipulator; and the above-described flexible-manipulator control device that controls the drive means on the basis of an operation amount input via an operation unit operated by an operator.

According to this aspect, when the operator operates the operation unit, a drive command signal for the drive means of the flexible manipulator is output from the flexible-manipulator control device in response to an operation amount input to the operation unit. In the flexible-manipulator control device, the control parameter is adjusted according to the curved state of the insertion portion, thereby making it possible to output an appropriate drive command signal to the drive means on the basis of the operation amount sent from the operation unit and to accurately control the movable part.

### REFERENCE SIGNS LIST

1 medical manipulator system
2 flexible-manipulator control device
3 master device (operation unit)
4 flexible manipulator
6 insertion portion
7 gripping portion (movable part)
8 drive portion (drive means)
11 identification-information input unit
12 storage unit (physical-information storage unit)
13 selection unit (physical-information selection unit)
14 position-information input unit
15, 18 drive control unit (drive control units A, B, and C, control units)
16 parameter adjustment unit
A operator
O patient

1. A flexible-manipulator control device that controls, according to a control parameter, a drive means of a flexible manipulator having a movable part at a distal end of a flexible insertion portion to be inserted into the body and having, at a proximal end thereof, the drive means for driving the movable part, the flexible-manipulator control device comprising:
a physical-information storage unit that stores physical information of a patient into which the insertion portion is inserted;
a position-information input unit to which is input position information of the movable part in a state in which it is inserted into the patient; and
a parameter adjustment unit that adjusts the control parameter on the basis of the physical information stored in the physical-information storage unit and the position information input to the position-information input unit.

2. A flexible-manipulator control device according to claim 1,
wherein the physical-information storage unit stores physical information of a plurality of patients in association with identification information of the patients;
the flexible-manipulator control device further comprises:
an identification-information input unit to which identification information of a patient is input; and
a physical-information selection unit that selects the physical information of the corresponding patient stored in the physical-information storage unit, on the basis of the identification information of the patient input to the identification-information input unit; and
the parameter adjustment unit adjusts the control parameter on the basis of the physical information selected by the physical-information selection unit and the position information input to the position-information input unit.

3. A flexible-manipulator control device according to claim 1, wherein the physical information is information about the shape of an insertion path in which the insertion portion is inserted into the body.

4. A flexible-manipulator control device according to claim 1, wherein the position information of the movable part is an insertion amount of the insertion portion inserted into the body.

5. A flexible-manipulator control device according to claim 4, wherein the position-information input unit is provided with a sensor for detecting the insertion amount of the insertion portion.

6. A flexible-manipulator control device according to claim 1, further comprising a plurality of control units in which different control parameters for controlling the drive means are set and that can be alternatively selected,
wherein the parameter adjustment unit switches between the control units on the basis of the physical information and the position information.

7. A flexible-manipulator control device according to claim 1, wherein the parameter adjustment unit estimates the shape of the insertion portion on the basis of the position information input to the position-information input unit and adjusts the control parameter on the basis of the estimated shape of the insertion portion.

8. A medical manipulator system comprising:
the flexible manipulator; and
a flexible-manipulator control device according to claim 1, that controls the drive means on the basis of an operation amount input via an operation unit operated by an operator.
